(19) United States
(12) Patent Application Publication
(10) Pub. No.: US 2010/0016430 A1
(43) Pub. Date: Jan. 21, 2010

(54) MODIFIED COCONUT OILS WITH BROAD ANTIMICROBIAL SPECTRUM

(75) Inventor: Kamariah Long, Selangor (MY)

Correspondence Address: MERCHANT & GOULD PC, P.O. BOX 2903, MINNEAPOLIS, MN, 55402-0903, US

(73) Assignee: MALAYSIAN AGRICULTERAL RESEARCH AND DEVELOPMENT IN, Selangor (MY)

(21) Appl. No.: 12/090,661

(22) PCT Filed: Nov. 16, 2006
(86) PCT No.: PCT/MY2006/000028

§ 371 Date: Apr. 18, 2008

(30) Foreign Application Priority Data
Dec. 7, 2005 (MY) PI 20055720

(51) Int. Cl. A61K 31/20; C07C 53/126; A61P 17/00

(52) U.S. Cl. 514/558; 554/1

(57) ABSTRACT

The present invention discloses antimicrobial compositions of modified coconut oil and palm kernel oil derived from catalytic activity of 1,3 positional specific lipases. Said modified oil compositions comprises of free fatty acids (>9.4%), monoglycerides (>1.3%), diglycerides (>22.8%) and triglycerides (>25%) which inhibits the growth of gram positive bacteria i.e. Staphylococcus aurous aureus, Listeria monocytogenes, Sterptococcus pyogene, gram negative bacteria i.e. Vibrio cholerae, Escherichia coli and yeast i.e. Candida albicans.

### FIELD OF THE INVENTION

This invention relates to modified oil having antimicrobial properties, in particular to modified coconut oil compositions which contain mixtures of medium chain fatty acids having 8 to 12 carbon atoms such as caprylic acid, capric acid, lauric acid and their corresponding monoglycerides. Said modified coconut oil is derived from catalytic activity of 1,3 positional specific lipase on coconut oil.

### BACKGROUND OF THE INVENTION

Medium chain free fatty acids and their corresponding monoglycerides have been found to have a broad spectrum of anti microbial activity against enveloped viruses and various bacteria in vitro (Kabara, 1978; Shibasaki and Kato, 1978; Welsh et al. 1979 Thormar et al., 1987; Isaacs et al. 1995), including human pathogens like herpes simplex virus (Thormar et al. 1987; Kristmundsdottir et al. 1999), Nesseria gonorrhoeae (Bergsson et al., 1999), Candida albicans (Bergsson et al, 2001), Chlamydia trachomatis (Bergsson et al., 1998), Helicobacter pylori (Bergsson et al, 2002) and Staphylococcus aureus (Kabara 1984). In addition, these compounds also known to have antimicrobial effect against food-borne pathogens like Listeria monocytogenes (Wang and Johnson, 1992), enterotoxigenic Escherichia coli (Petschow et al 1998) and Clostridium botulinum (Glass and Johnson, 2004). The mechanism by which these lipids kill bacteria is not known, but electron microscope studies indicate that they disrupt cell membrane permeability barrier ( Bergsson et al, 1998; Thormar et al. 1987).

Saturated medium chain fatty acid are fatty acids that have 8 to 12 carbon atoms (C₆ to C₁₂). Among the medium chain fatty acids, lauric acid and its corresponding monoglyceride, monolaurin, has been investigated extensively as an antimicrobial agent for foods and cosmetics (Shibasaki and Kato, 1978; Kabara, 1984). Lauric acid is a disease fighting agent that it is present in breast milk. The body converts lauric acid to a fatty acid derivative (monolaurin), which is the substance that protects infants from viral, bacterial or protozoal infections. Hierholzer and Kabara (1982) showed that monolaurin has virucidal effects on RNA and DNA viruses, which are surrounded by a lipid membrane.

The use of medium chain fatty acids and its corresponding monoester as anti microbial compound in several applications had been patented. i.e. monolaurin have been used in cleansing and conditioning hairs as well as the coat of animals (U.S. Pat. No. 5,378,731). This antimicrobial shampoo composition containing monolaurin which is safe for human and animal use. Monolaurin can also widely employed in toiletries and household articles, which need antifungal properties (U.S. Pat. Nos. 5,569,461 and 5,658,584). In addition its can be combined with bacteriocin i.e. nisin for the treatment of bacterial infections of the genus Helicobacter that cause various gastrointestinal disease including gastritis and ulcer (U.S. Pat. No. 5,660,842: U.S. Pat. No. 5,804,549). These fatty acids and their derivative thereof were also claims as a dietary supplement that controlling or reducing human's weight. (U.S. Pat. No. 6,054,480). Additionally these compounds and their monoester can be used to reduce the microbial contamination of processed meat and is particularly related to a product and a process to disinfect poultry carcasses. Moreover its help to kill harmful microbes on the under of a milk-producing animal (U.S. Pat. No. 6,699,907). They are also effective course of treatment for skin, mucous membrane and hair lesion (U.S. Pat. No. 5,208,257). The latest invention showed that with these fatty acids could be a therapeutic agents for the treatment of Alzheimer's disease and other diseases associated with reduced neuronal metabolism, including Parkinson' disease, Huntington's disease and epilepsy (U.S. Pat. No. 6,835,750)

Previous works have shown that susceptibility to medium chain fatty acids varies considerably among species, certain microbes were sensitive to certain fatty acids and monoglycerides. For example, Bergsson et. al 1998 showed that lauric acid, capric acid and monocaprin caused a greater than 10,000-fold reduction in the infectivity titer. When the fatty acids and monoglycerides were further compared at low concentration and shorter exposure times, lauric acid was more active than capric acid and monocaprin, causing a greater than 100,000 fold inactivation of C. trachomatis at a concentration of 5 mM for 5 min. Compare with monocaprin, monolaurin and monocaprylin at 10 mM concentration had negligible effect on C. trachomatis. In other worked done by Bergsson et al. (2002) found that none of the medium chain fatty acids (C₈, C₁₀ and C₁₂) and its monoglycerides derivatives showed significant antibacterial activity against Salmonella spp and E. Coli. However, in this experiment they found out that monocaprin and monolaurin at 10 mM concentration for 30 min at 37° C. proved to be the most active against H. pylori. Incorporation of monolaurin at 250 and 500 ppm into naturally contaminated cottage cheese was resulted in more than 90% inhibition of both Pseudomonas spp and coliform (Bautista et al., 1993). In the case of C. albicans, capric acid and lauric causes the fastest and most effective killing compare with its derivatives monoglycerides (Bergsson et al. 2001). However, myristic acid, palmitoleic acid, oleic acid and its derivates monoglycerides do not shown antimicrobial effect.

It seems that the ability of these medium chain fatty acids and their monoglycerides to act as antimicrobial agents are varied. Effectiveness of these compounds are always depending on their concentrations and the type of microbes involved. So far, test for antimicrobial activity of medium chain fatty acids and their corresponding monoglycerides were tested individually. Test was performed either using the lauric or capric or caprylic acid or their corresponding monoglycerides. The synergistic effect of both medium chain fatty acids and their respective monoglycerides in oil medium never being conducted and reported.

### SUMMARY OF THE INVENTION

It is therefore an object of the invention to provide for modified coconut oil derived form catalyzing coconut oil with 1,3-specific lipase, said modified coconut oil contains effective amounts of medium-chain fatty-acids and their corresponding monoglycerides, wherein said modified oil has antimicrobial properties. Said modified coconut oil having medium-chain fatty-acids comprises of caprylic acid (C₈), capric acid (C₁₀) and lauric acid (C₁₂).

### DETAILED DESCRIPTION OF THE INVENTION

In accordance with the object of the invention, efforts have been done in this work to get profiles of the modified coconut oil that contain medium chain fatty acids (special emphasis on C₈-C₁₂) and its respective monoglycerides which have antimicrobial activities towards bacteria, yeast, fungi and viruses. Coconut oil was selected because by natures it was rich source of medium chain fatty acids. It contains 90% saturated fatty acids, and of these, 45-48% is lauric acid and 30-36% are other short- and medium chain fatty acids. Preliminary work showed that coconut oil in the present form don't have antimicrobial activities (contains 7% diacylglycerol and remaining is the triglycerides). Analysis by HPTLC showed that fatty acids and monoglycerides are detected in the oil compositions. The invention also includes methods using 1,3 positional specific lipase to obtain not only oils that have antimicrobial properties but also effective and powerful antimicrobial agents.

According to the present invention 1,3 positional specific lipases is used to modify the coconut oil under specific reaction conditions to obtain profiles of saturated medium chain fatty acids and their respective monoglycerides that have broad antimicrobial spectrum towards bacteria and yeast. The modified coconut oil mixtures preferably contain high amount free fatty acids (C₈-C₁₂) and their corresponding monoglycerides that can be obtained through partial hydrolysis or glycerolysis reaction. The enzyme used in the present invention is an enzyme such as lipase, and preferably immobilized onto a suitable enzyme carrier. The said specific lipase possess 1,3-position i.e. Lipozyme TL IM (Rhizomucor miehei). Reactions to obtain modify oils were as followed.

Reaction 1—Modified 1 and Modified 2

Enzymatic reaction was carried out using 2.5 g 1,3-positional specific lipases with 250 g coconut oil and 2.5 ml distilled water. The reaction was conducted at 45° C. at 250 r.p.m. Samples were withdrawn for analysis after 24 h reaction (Modified 1) and 120 h reaction (Modified 2). Samples were then passed through funnel containing sodium sulfate powder to remove water form the sample. The reaction mixture was centrifuged to separate the oil phase. High performance thin layer chromatography technique and gas chromatography technique were carried out to determine the lipid classes and fatty acids compositions of the oil sample respectively. The modified oils were then analyzed for their antimicrobial activities of following Test 1: Minimal Microcidal Concentration (MMC, >90%) and Test 2: Time-kill studies

Reaction 2—Modified 3

Twenty ml of coconut oil was added into a 125 ml flask containing 8 g glycerol and 160 ul of distilled water. The reaction mixtures were incubated at 35° C., 300 r.p.m for a time until the incubation temperature reached 35° C. Lipozyme TL IM at 250 mg was then added into the reaction mixture and incubated for 24 h at 35° C. Consequently, the oil sample was then subjected at 25° C. for another 3 days.

Reaction 3—Modified 4

A mixture of 30.4 g glycerol, 1.09 ml of water, 1 g of Lipozyme TL IM and 100 g of coconut oil was prepared. The reaction mixture was first incubated at 30° C. for 6 h with constant stirring at 800 r.p.m. The mixture was then transferred to 5° C. for up to 3 days before analyze.

Reaction 4—Modified 5

The reaction mixture was prepared according to Modified 4. The reaction was carried out at 30° C., 800 r.p.m. for 16 h. before analyze.

Reaction 5—Modified 6

Twenty ml oil from Modified 2 was added into a mixture containing 8 g glycerol, and 160 μl sterile distilled water. The reaction mixture was pre incubated at 35° C., 300 r.p.m. The reaction was initiated by adding 250 mg Lipozyme TL IM into the mixture and reaction was carried for 24 h. Consequently the mixture was then incubated at 25° C. for 3 days

Test 1: Minimal Microcidal Concentration (MMC, >90% kill)

All well were inoculated with 120 μl broth (BHI, contain 0.1% Tween 80, for Gram-positive bacteria, TSB for Gram-negative bacteria; PDB for yeast). 120 μl of antimicrobial agent was inoculated into first well. From first well, 120 μl of the mixture was transferred into the second well and so on until the 12^{th} well. Inoculum which was adjusted to 10⁵-10⁶ cfu/ml was inoculated into each well. The plates were incubated at 37° C. (2 days) for bacteria and 32° C. (3 days) for yeast. Results were expressed in terms of MMC₉₀ (minimal bactericidal concentration, >90% killing) as shown in Table 1.

Test 2: Time-Kill Studies

Inocula were developed by inoculation of a loopful cell in 50 ml broth (BHI for Gram-positive bacteria; TSB for Gram-negative bacteria; PDB for yeast) in flask and shaked at optimum temperature overnight. This was used to inoculate BHI, TSB or PDB broth that contained 50% of filtered sterilized treated VCO. The initial inoculum was adjusted to 10⁴-10⁶ cfu/ml. At time interval, 1 ml of reaction mixture was withdrawn and serial dilution was carried out in ringer solution. Viable colony was enumerated by plating dilutions on plate count agar (PCA). The plates were incubated at 37° C. (2 days) for bacteria and 32° C. (3 days) for yeast. A control experiment was done in the presence of 50% sterile distilled water.

TABLE 1
Minimum Microcidal Concentration (MMC₉₀) of modified virgin
coconut oils against pathogenic microorganisms

| | Strain | | | | | |
|---|---|---|---|---|---|---|
| MBC, mg/ml | S. aureus | L. monocytogenes | C. albicans | E. coli | S. pyogenes | P. acne |
| Untreated | none | none | none | none | none | none |
| Virgin Coconut | | | | | | |
| Oil | | | | | | |
| Modified 1 | 156.25 | 156.25 | 156.25 | none | 156.25 | none |
| Modified 2 | 78.13 | 78.13 | 78.13 | none | 78.13 | none |
| Modified 3 | 4.88 | 4.88 | 2.44 | 2500 | 4.88 | none |
| Modified 4 | 78.13 | 78.13 | 2.44 | 2500 | 78.13 | none |
| Modified 5 | 78.13 | 78.13 | 4.88 | 2500 | 78.13 | none |
| Modified 6 | 4.88 | 4.88 | 2.44 | 2500 | 4.88 | none |

TABLE 2
Inhibition of pathogenic microorganisms by modified virgin coconut oils evaluated by
time-kill studies

| | Modified | Number of viable bacteria (log10 cfu/ml) | | | | | | |
|---|---|---|---|---|---|---|---|---|
| | Virgin | at time interval (hours) | | | | | | |
| Microbes | Coconut Oil | 0 | 2 | 4 | 6 | 8 | 24 | 48 |
| S. aureus | Modified 1 | 5.69 | 5 | 3.6 | 3.11 | 2.62 | 0 | 0 |
| | Modified 2 | 6.32 | 0 | 0 | 0 | 0 | 0 | 0 |
| | Modified 3 | 6.27 | 0 | 0 | 0 | 0 | 0 | 0 |
| | Modified 4 | 6.04 | 4.25 | 0 | 0 | 0 | 0 | 0 |
| | Modified 5 | 6.08 | 3.96 | 0 | 0 | 0 | 0 | 0 |
| | Modified 6 | 5.87 | 0 | 0 | 0 | 0 | 0 | 0 |
| L. monocytogenes | Modified 1 | 5.71 | 5.02 | 3.62 | 3.15 | 2.66 | 0 | 0 |
| | Modified 2 | 6.28 | 0 | 0 | 0 | 0 | 0 | 0 |
| | Modified 3 | 6.15 | 0 | 0 | 0 | 0 | 0 | 0 |
| | Modified 4 | 6.06 | 4.25 | 0 | 0 | 0 | 0 | 0 |
| | Modified 5 | 6.08 | 3.76 | 0 | 0 | 0 | 0 | 0 |
| | Modified 6 | 5.34 | 0 | 0 | 0 | 0 | 0 | 0 |
| C. albicans | Modified 1 | 4.59 | 4.61 | 4.54 | 4.44 | 4.29 | 2.81 | 2.71 |
| | Modified 2 | 6.48 | 5.56 | 5.48 | 5.65 | 5.38 | 2.16 | 2 |
| | Modified 3 | 5.76 | 4.98 | 3.21 | 3.45 | 3.11 | 0 | 0 |
| | Modified 4 | 5.98 | 5.65 | 5.15 | 4.18 | 3.46 | 0 | 0 |
| | Modified 5 | 6.02 | 5.68 | 5.43 | 4.75 | 3.86 | 0 | 0 |
| | Modified 6 | 5.98 | 4.13 | 3.06 | 2.99 | 2.14 | 0 | 0 |
| E. coli | Modified 1 | ND | ND | ND | ND | ND | ND | ND |
| | Modified 2 | 6.32 | 6.48 | 7.52 | 8.34 | 9.16 | 10.27 | 11.16 |
| | Modified 3 | 6.21 | 6.54 | 5.82 | 5.96 | 4.32 | 2.58 | 2.69 |
| | Modified 4 | 6.01 | 6.26 | 5.64 | 5.28 | 5.04 | 2.96 | 2.71 |
| | Modified 5 | 6.02 | 6.15 | 6.01 | 5.78 | 5.44 | 2.84 | 2.56 |
| | Modified 6 | 6.14 | 5.28 | 5.35 | 6.02 | 4.67 | 2.64 | 2.63 |
| S. pyogenes | Modified 1 | 5.48 | 5.21 | 4.23 | 3.08 | 2.12 | 0 | 0 |
| | Modified 2 | 6.1 | 0 | 0 | 0 | 0 | 0 | 0 |
| | Modified 3 | 5.7 | 0 | 0 | 0 | 0 | 0 | 0 |
| | Modified 4 | 6.01 | 4.68 | 0 | 0 | 0 | 0 | 0 |
| | Modified 5 | 6.04 | 4.74 | 0 | 0 | 0 | 0 | 0 |
| | Modified 6 | 5.49 | 0 | 0 | 0 | 0 | 0 | 0 |
| V. cholerae | Modified 2 | 6.34 | 0 | 0 | 0 | 0 | 0 | 0 |

TABLE 3
Inhibition of pathogenic microorganisms by modified palm kernel oil
evaluated by kill studies

| | Number of viable bacteria (log cfu/ml) at time interval | | | | | | |
|---|---|---|---|---|---|---|---|
| | (hours) | | | | | | |
| Microbe | 0 | 2 | 4 | 6 | 8 | 24 | 48 |
| S. aureus | 7.28 | 7.24 | 7.18 | 7.18 | 5.31 | 5.85 | 0 |
| E. coli | 6.93 | 7.30 | 10.76 | 12.10 | 13.56 | 10.22 | 8.56 |
| C. albicans | 5.71 | 6.03 | 5.92 | 5.95 | 5.87 | 5.98 | 5.74 |

The following profiles of modified virgin coconut oils and palm kernel oil have been tested for their antimicrobial activities and found to be active against a substantial group of microorganisms

MODFIED 1- Profile 1

| | Percentages | | |
|---|---|---|---|
| | (% area by | | Concentration |
| Lipid Classes | HPTLC) | Medium chain length | (mg/g) |
| Fatty acids | 10.58 | Caprylic acid (C₈) | 10.44 |
| | | Capric acid (C₁₀) | 7.27 |
| | | Lauric acid (C₁₂) | 54.61 |
| Monoglycerides | 1.32 | Monocaprylin | 0.67 |
| | | Monocaprin | 0.42 |
| | | Monolaurin | 20.96 |
| Diglycerides | 22.81 | | |
| Triglycerides | 65.29 | | |

MODIFIED 2- Profile 2

| | Percentages | | |
|---|---|---|---|
| | (% area by | | Concentration |
| Lipid classes | HPTLC) | Medium chain length | (mg/g) |
| Fatty acids | 14.13 | Caprylic acid (C₈) | 13.95 |
| | | Capric acid (C₁₀) | 9.89 |
| | | Lauric acid (C₁₂) | 70.54 |
| Monoglycerides | 1.51 | Monocaprylin | 1.30 |
| | | Monocaprin | 0.50 |
| | | Monolaurin | 27.00 |
| Diglycerides | 26.88 | | |
| Triglycerides | 57.48 | | |

MODIFIED 3-Profile 3

| | Percentages | | |
|---|---|---|---|
| | (% area by | | Concentration |
| Lipid Classes | HPTLC) | Medium chain length | (mg/g) |
| Fatty acids | 23.40 | Caprylic acid (C8) | 23.10 |
| | | Capric acid (C10) | 16.08 |
| | | Lauric acid (C12) | 116.81 |
| Monoglycerides | 14.28 | Monocaprylin | 16.04 |
| | | Monocaprin | 10.35 |
| | | Monolaurin | 75.54 |
| Diglycerides | 37.24 | | |
| Triglycerides | 25.08 | | |

MODIFIED 4- Profile 4

| | Percentages | | |
|---|---|---|---|
| | (% by | | Concentration |
| Lipid Classes | HPTLC) | Medium chain length | (mg/g) |
| Fatty acids | 13.44 | Caprylic acid (C₈) | 13.35 |
| | | Capric acid (C₁₀) | 9.34 |
| | | Lauric acid (C₁₂) | 68.61 |
| Monoglycerides | 8.04 | Monocaprylin | 8.00 |
| | | Monocaprin | 5.40 |
| | | Monolaurin | 41.10 |
| Diglycerides | 31.36 | | |
| Triglycerides | 47.16 | | |

MODIFIED 5- Profile 5

| | Percentages | | |
|---|---|---|---|
| | (% area by | | Concentration |
| Lipid Classes | HPTLC) | Medium chain length | (mg/g) |
| Fatty acids | 9.40 | Caprylic acid (C₈) | 9.40 |
| | | Capric acid (C₁₀) | 6.56 |
| | | Lauric acid (C₁₂) | 71.77 |
| Monoglycerides | 12.10 | Monocaprylin | 12.25 |
| | | Monocaprin | 8.05 |
| | | Monolaurin | 71.77 |
| Diglycerides | 39.98 | | |
| Triglycerides | 38.47 | | |

MODIFIED 6- Profile 6

| | Percentages | | |
|---|---|---|---|
| | (% area by | | Concentration |
| Lipid Classes | HPTLC) | Medium chain length | (mg/g) |
| Fatty acids | 25.01 | Caprylic acid (C₈) | 24.63 |
| | | Capric acid (C₁₀) | 17.81 |
| | | Lauric acid (C₁₂) | 133.70 |
| Monoglycerides | 11.45 | Monocaprylin | 11.82 |
| | | Monocaprin | 8.29 |
| | | Monolaurin | 57.16 |
| Diglycerides | 35.98 | | |
| Triglycerides | 27.56 | | |

Modified Palm Kernel Oil

| | Percentages | | |
|---|---|---|---|
| | (% area by | | Concentration |
| Lipid Classes | HPTLC) | Medium chain length | (mg/g) |
| Fatty acids | 12.87 | Caprylic acid (C₈) | 16.10 |
| | | Capric acid (C₁₀) | 13.06 |
| | | Lauric acid (C₁₂) | 58.19 |
| Monoglycerides | 1.31 | Monocaprylin | 1.64 |
| | | Monocaprin | 2.03 |
| | | Monolaurin | 19.71 |
| Diglycerides | 21.66 | | |
| Triglycerides | 64.16 | | |

According to the invention, methods to obtain antimicrobial compositions containing i.e. medium chain fatty acids and their corresponding monoester through partial hydrolysis and glyecrolysis of coconut oil are provided. Modified oil 1 and Modified oil 2 were obtained from hydrolysis reaction of virgin coconut oil at 24 h and 120 h, respectively. Test for antimicrobial activities as MMC₉₀ (Table 1) and time-kill studies (Table 2) showed that the Modified 2 have more powerful antimicrobial activities compare to Modified 1. This is probably because of high amount of fatty acids (14%) present in Modified 2 (Profile 2). The amount of saturated medium chain fatty acids especially caprylic, capric and lauric (mg/g) oil were increased (Profile 2). However, it was noticed that modified oil compositions from profile 1 and profile 2 can't stopped the growth of E. coli and P. acne. Obviously, from the time-kill studies C. albicans growth was not 100% inhibited even after 48 h incubation (Table 2). Modified 1 and Modified 2 were effective toward gram positive bacteria where 100% inhibitions were noted after 8 h and 2 h incubation, respectively. Preliminary results indicated that the amount fatty acids i.e. C₈, C₁₀ and C₁₂ might play important role in inactivation of the growth of gram positive bacteria. Modified 3, 4, 5 and 6 were prepared differently from Modified 1 and 2. They were obtained through glycerolysis reactions. In all reactions glycerol and Lipozyme TL IM were added.

In comparison, Modified 3, 4, 5 and 6 were more effective in killing C. albicans than Modified 1 and 2. Hundred percent inhibitions were noted after 8 hours incubation. Minimal Microbial Concentration towards C. albicans was obtained at 2.44 mg/ml whereas Modified 5 need higher concentration e.g. 4.88 mg/ml. Among the modified oil samples, Modified 3 and Modified 6 contained powerful antimicrobial activities. In addition it was noted that MMC₉₀ for gram positive bacteria like S. aureus, L. monocytogenes, S. pyogenes of Modified 3, 4, 5 and 6 were lower than the Modified 1 and 2. The most interesting thing is that these modified coconut oils have antimicrobial activity towards the gram negative, E. coli although the MMC₉₀ required is still higher compare to the gram positive bacteria. Modified 3 and 6 were also proved to be more potent toward gram positive bacteria compare to Modified 4 and 5. Detail analysis of the lipid classes of the Modified 3, 4, 5 and 6 showed that high amount of fatty acids and monoglycerides content play important role in broaden the antimicrobial spectrum of these modified oils. About 8% to 14% monoglycerides were required to control and reduce the growth of E. coli and 100% inhibition of C. albicans. Whereas high amount of fatty acids in Modified 3 and 6 make the modified oils more potent towards gram positive bacteria (Profile 3 and Profile 6). The MMC₉₀ for all gram positive bacteria for Modified 3 and 6 were at 4.88 mg/ml which are sixteen times lower than that of Modified 4 and 5. Fatty acids together with monoglycerides have a synergistic effect that inhibited 100% growth of C. albicans.

Palm kernel oil, which fatty acid composition similar to coconut oil, can also be modified using 1,3 specific lipase. The modified palm kernel oil was also found to have antimicrobial property towards S. aureus. Total inhibition of S. aureus was noted after 24 h exposure. On the other hand it was noted that the amount of medium chain fatty acid and it corresponding fatty acids in the modified palm kernel oil didn't significant to stop the growth or kill the E. coli and C. albicans

Modified oils compositions in the present invention were found to exhibit good shelf stability against oxidation and can be safely used to combat bacteria, yeast and viruses that affect human, as food preservatives, products for personal hygiene and prevention of skin infection.

As will be apparent to those skilled in the art in the light of the foregoing disclosure, many alterations and modifications are possible in the practice of this invention without departing from the scope thereof. Accordingly, the scope of the invention is to be construed in accordance with the substance defined by the following claims.

### REFERENCES CITED

Kabara, J. J. (1978) “Fatty acids and dertivatives as antimicrobial agents.” In J. J. Kabara (ed) The Pharmacological Effect of Lipids. American Oil Chemists' Society, Champaign Ill., pp: 1-14.

Shibasaki, I. & Kato, N. (1978) “Combined effects on anti-bacterial activity of fatty acids and their esters against gram-negative bacteria.” In J. J. Kabara (ed) The Pharmacological Effect of Lipids. American Oil Chemists' Society, Champaign, Ill., pp: 15-23.

Welsh, J. K, Arsenakis, M., Coelen, R. J. & May, J. T. (1979) “Effect of antiviral lipids, heat, and freezing on the activity of viruses in human milk.” Journal of Infectious Disease. 140:322-328.

Thormar, H., Isaacs, C., Brown, H. R., Barshatzky, M. R. & Pessolano, T. (1987) “Inactivation of enveloped viruses and killing of cells by fatty acids and monoglycerides.” Antimicrobial Agents and Chemotherapy. 31:27-31.

Isaacs, C. E., Litov, R. E. & Thormar, H. (1995) “Antimicrobial activity of lipids added to human milk, infant formula, and bovine milk.” Nutritional Biochemistry. 6:362-366.

Kristmundsdóttir, T., Árnadóttir, S., Bergsson, G. & Thormar, T. (1999) “Development and evaluation of microbicidal hydrogels containing monoglycerides as the active ingredients.” Journal of Pharmacological Science. 88:1011-1015.

Bergsson, G., Steingrímsson, Ó. & Thormar, H. (1999) “In vitro susceptibilities of Neisseria gonorhoeae to fatty acids and monoglycerides.” Antimicrobial Agents and Chemotherapy. 43:2790-2792.

Bergsson, G., Arnfinnsson, J., Steingrímsson, Ó. & Thormar, H. (2001) “In vitro killing of Candida albicans by fatty acids and monoglycerides.” Antimicrobial Agents and Chemotherapy. 45(11):3209-3212.

Bergsson, G., Arnfinnsson, J., Karlsson, S. M., Steingrímsson, Ó. & Thormar, H. (1998) “In vitro inactivation of Chlamydia trachomatis by fatty acids and monoglycerides.” Antimicrobial Agents and Chemotherapy. 42(9):2290-2294.

Bergsson, G., Steingrímsson, Ó. & Thormar, H. (2002) “Bactericidal effects of fatty acids and monoglycerides on Helicobacter pylori.” International Journal of Antimicrobial Agents. 20:258-262.

Kabara, J. J. (1984) “Antimicrobial agents derived from fatty acids.” Journal of American Oil Chemists' Society. 61(2):397-403.

Wang, L. L. & Johnson, E. A. (1992) “Inhibition of Listeria monocytogenes by fatty acids and monoglycerides.” Applied and Environmental Microbiology. 58(2):624-629.

Petschow, B. W., Batema, R. P., Talbott, R. D. & Ford, L. L. (1998) “Impact of medium-chain monoglycerides on intestinal colonization by Vibrio cholerae or enterotoxigenic Escherichia coli.” Journal of Medicinal Microbiology. 47:383-389.

Glass, K. A. & Johnson, E. A. (2004) “Antagonistic effect of fat on the antibotulinal activity of food preservatives and fatty acids.” Food Microbiology. 21:675-682.

Hierholzer, J. C. & Kabara, J. J. (1982) “In vitro effects of monolaurin compounds on enveloped RNA and DNA viruses.” Journal of Food Safety. 4:1-12.

Bautista, D. A., Durisin, M. D., Razavi-Rohani, S. M., Hill, A. R. & Griffiths, M. W. (1993) “Extending the shelf-life of cottage cheese using monolaurin.” Food Research International. 26:203-208.

1. Modified coconut oil derived form catalyzing coconut oil with 1,3-specific lipase, said modified coconut oil contains effective amounts of medium-chain fatty-acids and their corresponding monoglycerides, wherein said modified oil has antimicrobial properties.

2. Modified coconut oil according to claim 1 wherein said of medium-chain fatty-acids comprises caprylic acid (C₈), capric acid (C₁₀) and lauric acid (C₁₂).

3. Modified coconut oil according to claim 1 wherein said corresponding monoglycerides comprises monocaprylin, monocaprin and monolaurin.

4. Modified coconut oil according to claim 1 wherein said modified coconut oil contains more than 10% concentration of free fatty-acids and more than 1% concentration of monoglycerides.

5. Modified coconut oil according to claim 1 wherein said 1,3-specific lipase is obtained from Rhizomucor miehei.

6. Modified coconut oil according to claim 1 wherein said modified coconut oil is derived from catalytic activity of 1,3-specific lipase on coconut oil by partial hydrolysis.

7. Modified coconut oil according to claim 6 wherein said modified oil is subjected to glycerolysis reaction after said partial hydrolysis reaction.

8. Modified coconut oil according to any one of claim 1 wherein said coconut oil is virgin coconut oil.

9. Modified coconut oil according to claim 1 wherein said modified coconut oil contains more than 10% concentration of free fatty-acids and less than 2% concentration of monoglycerides.

10. Modified coconut oil according to claim 9 wherein said free fatty-acids comprises more than 10 mg/g caprylic acid (C₈), more than 7 mg/g capric acid (C₁₀) and more than 54 mg/g lauric acid (C₁₂).

11. Modified coconut oil according to claim 9 wherein said monoglycerides comprises more than 0.6 mg/g monocaprylin, more than 0.4 mg/g monocaprin and more than 20 mg/g monolaurin.

12. Modified coconut oil according to claim 1 wherein said modified coconut oil is effective against gram-positive bacteria such as S. aureus, L. monocytogenenes and S. pyogenes.

13. Modified coconut oil according to claim 1, wherein said modified coconut oil is effective against gram-negative bacteria such as V. cholerae.

14. Modified coconut oil according to claim 1, wherein said modified coconut oil is effective against yeast such as C. albicans.

15. Modified coconut oil according to claim 9, wherein said modified coconut oil is derived from catalytic activity of 1,3-specific lipase on coconut oil by partial hydrolysis reaction.

16. Modified coconut oil according to claim 15 wherein said 1,3-specific lipase is obtained from Rhizomucor miehei.

17. Modified coconut oil according to any one of claim 15 wherein said coconut oil is virgin coconut oil.

18. Modified coconut oil according to claim 1 wherein said modified coconut oil contains more than 10% concentration of free fatty-acids and more than 8% concentration of monoglycerides.

19. Modified coconut oil according to claim 18 wherein said free fatty-acids comprises more than 9 mg/g caprylic acid (C₈), more than 6 mg/g capric acid (C₁₀) and more than 68 mg/g lauric acid (C₁₂).

20. Modified coconut oil according to claim 18 wherein said monoglycerides comprises more than 8 mg/g monocaprylin, more than 5 mg/g monocaprin and more than 41 mg/g monolaurin.

21. Modified coconut oil according to claim 1, wherein said modified coconut oil is more effective against gram-positive bacteria such as S. aureus, L. monocytogenenes and S. pyogenes.

22. Modified coconut oil according to claim 1, wherein said modified coconut oil is more effective against yeast such as C. albicans.

23. Modified coconut oil according to claim 22 wherein said modified coconut oil inhibits 100% C. albicans within 8 hr incubation.

24. Modified coconut oil according to claim 1, wherein said modified coconut oil is effective in reducing growth of gram negative bacteria such as E. coli.

25. Modified coconut oil according to claim 1, wherein said modified coconut oil is derived from catalytic activity of 1,3-specific lipase on coconut oil by partial hydrolysis followed by glycerolysis reaction.

26. Modified coconut oil according to claim 25 wherein said 1,3-specific lipase is obtained from Rhizomucor miehei.

27. Modified coconut oil according to any one of claim 25 wherein said coconut oil is virgin coconut oil.

28. The use of modified coconut oil according to claim 1 for treatment and prevention of skin diseases.

29. The use of modified coconut oil according to claim 1 for food preservation.

30. The use of modified coconut oil according to claim 1, for formulation in personal hygiene products or incorporated as part of the formulation of said personal hygiene products.

31. The use of modified coconut oil according to claim 1, as an antibacterial agent wherein the dosage form is a capsule.

32. The use of modified coconut oil according to claim 1 as a natural antibiotic agent for preventing disease in animal.

33. Modified palm kernel oil derived form catalyzing palm kernel oil with 1,3-specific lipase, said modified oil contains effective amounts of medium-chain fatty-acids and their corresponding monoglycerides, wherein said modified oil has antimicrobial properties.

34. Modified oil according to claim 33 wherein said modified oil contains more than 10% concentration of free fatty-acids and more than 1% concentration of monoglycerides.

35. Modified oil according to claim 33 wherein said 1,3-specific lipase is obtained from Rhizomucor miehei.

36. The use of modified oil according to claim 33 for treatment and prevention of skin diseases.

37. The use of modified oil according to claim 33 for food preservation.

38. The use of modified oil according to claim 33 for formulation in personal hygiene products or incorporated as part of the formulation of said personal hygiene products.

39. The use of modified oil according to claim 33 as an antibacterial agent wherein the dosage form is a capsule.

40. The use of modified oil according to claim 33 as a natural antibiotic agent for preventing disease in animal.
